(19) United States
(12) Patent Application Publication
(10) Pub. No.: US 2013/0090578 A1
(43) Pub. Date: Apr. 11, 2013

(54) DEVICE AND METHODS FOR RENAL NERVE MODULATION

(71) Applicant: BOSTON SCIENTIFIC SCIMED, INC., Maple Grove, MN (US)

(72) Inventors: Scott R. SMITH, Chaska, MN (US); Leonard B. RICHARDSON, Brooklyn Park, MN (US); Roger N. HASTINGS, Maple Grove, MN (US)

(73) Assignee: BOSTON SCIENTIFIC SCIMED, INC., Maple Grove, MN (US)

(21) Appl. No.: 13/647,968

(22) Filed: Oct. 9, 2012

Related U.S. Application Data
(60) Provisional application No. 61/545,413, filed on Oct. 10, 2011.

(51) Int. Cl. A61N 7/00

(52) U.S. Cl. 601/2

(57) ABSTRACT

Systems for nerve modulation are disclosed. An example system may include a first elongate element having a distal end and a proximal end and having at least one transducer disposed adjacent the distal end. The transducer may be an ultrasound transducer. Activation of the transducer may radiate acoustic energy from in two directions simultaneously.

### CROSS-REFERENCE TO RELATED APPLICATIONS

This application claims priority under 35 U.S.C. §119 to U.S. Provisional Application Ser. No. 61/545,413, filed Oct. 10, 2011, the entirety of which is incorporated herein by reference.

### TECHNICAL FIELD

The present disclosure relates to methods and apparatuses for nerve modulation techniques such as ablation of nerve tissue or other destructive modulation techniques through the walls of blood vessels.

### BACKGROUND

Certain treatments require the temporary or permanent interruption or modification of select nerve function. One example treatment is renal nerve ablation which is sometimes used to treat conditions related to congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and thus can be accessed intravascularly through the walls of the blood vessels. In some instances, it may be desirable to ablate perivascular renal nerves using ultrasound energy. However, some ultrasound treatments may not utilize energy efficiently and may require cooling. It may be desirable to provide for alternative systems and methods for intravascular nerve modulation.

### SUMMARY

The disclosure is directed to several alternative designs, materials and methods of manufacturing medical device structures and assemblies for partially occluding a vessel and performing nerve ablation.

Accordingly, one illustrative embodiment is a system for nerve modulation that may include an elongate shaft having a proximal end region and a distal end region. An ultrasound transducer including a first side surface and a second side surface may be positioned adjacent to the distal end region of the elongate shaft. The ultrasound transducer may further include a retaining ring disposed about the perimeter of the transducer and a post attached to the retaining ring. The transducer may be attached to the elongate shaft via the retaining ring and post. The transducer may include a matching layer disposed on both the first side surface and the second side surface and may radiate acoustic energy in two directions simultaneously. The above summary of an example embodiment is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 is a perspective view of a distal end of an illustrative renal nerve modulation system.

FIG. 3 is a cross-section of the illustrative renal nerve modulation system shown in FIG. 2.

FIG. 4 is a perspective view of a distal end of another illustrative renal nerve modulation system.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

While the devices and methods described herein are discussed relative to renal nerve modulation, it is contemplated that the devices and methods may be used in other applications where nerve modulation and/or ablation are desired, such as, but not limited to: blood vessels, urinary vessels, or in other tissues via trocar and cannula access. In some instances, it may be desirable to ablate perivascular renal nerves with ultrasound ablation.

Ultrasound ablation may be a faster and less expensive alternative to radiofrequency (RF) ablation. However, a traditional transducer may waste energy as energy is directed in one direction by a backing layer. In some instances, the backing layer may reflect most of the acoustic energy such that the acoustic energy is directed out a single side of the transducer, but may also produce some additional losses resulting in transducer heating. The backing layer may also block heat conduction for cooling from the backing layer side of the transducer, thus only allowing cooling from a single side. A transducer formed without the backing layer may allow for bidirectional ablation, improve efficiency, and allow for better heat transfer for transducer cooling.

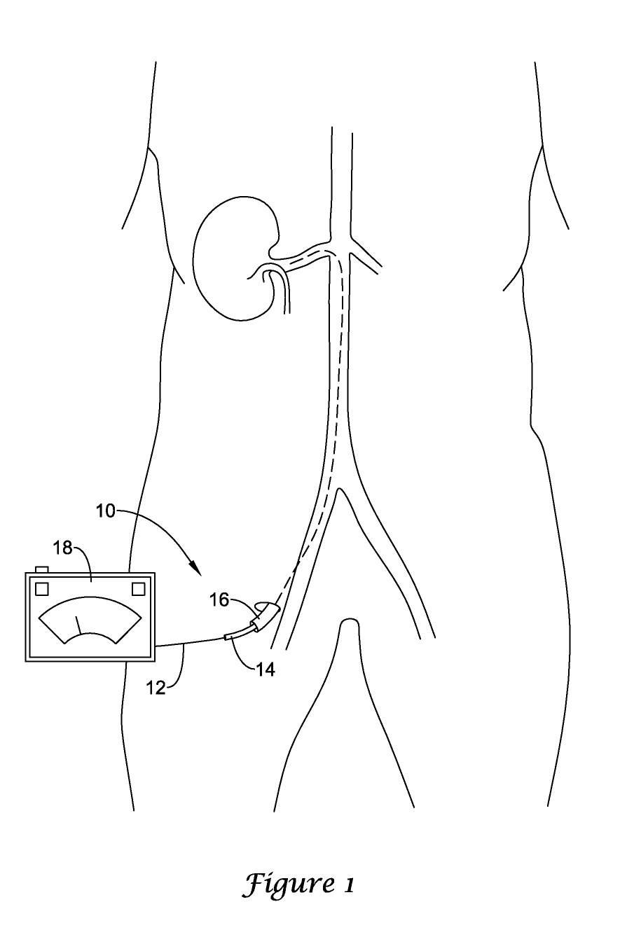

FIG. 1 is a schematic view of an illustrative renal nerve modulation system 10 in situ. System 10 may include an element 12 for providing power to a transducer disposed adjacent to, about, and/or within a central elongate shaft 14 and, optionally, within a sheath 16, the details of which can be better seen in subsequent figures. A proximal end of element 12 may be connected to a control and power element 18, which supplies the necessary electrical energy to activate the one or more transducers at or near a distal end of the element 12. The control and power element 18 may include monitoring elements to monitor parameters such as power, temperature, voltage, and/or frequency and other suitable parameters as well as suitable controls for performing the desired procedure. In some instances, the power element 18 may control an ultrasound transducer. The transducer may be configured to operate at a frequency of approximately 9-10 megahertz (MHz). It is contemplated that any desired frequency may be used, for example, from 1-20 MHz. However, it is contemplated that frequencies outside this range may also be used, as desired.

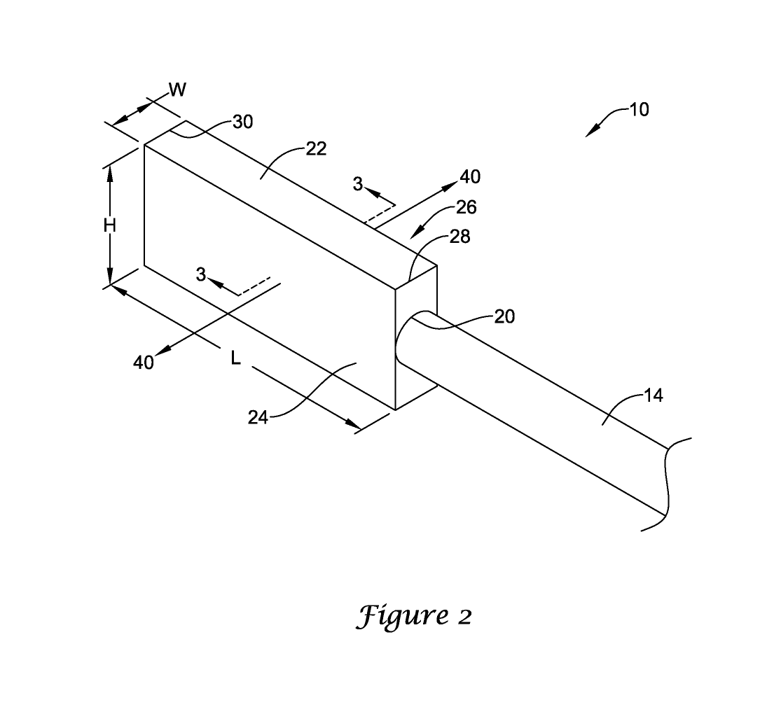

FIG. 2 is a perspective view of a distal end of an illustrative renal nerve modulation system 10. The system 10 may include an elongate shaft 14 having a distal end 20. The elongate shaft 14 may extend proximally from the distal end 20 to a proximal end (not shown) configured to remain outside of a patient's body. The proximal end of the elongate shaft 14 may include a hub attached thereto for connecting other diagnostic and/or treatment devices or for providing a port for facilitating other interventions.

It is contemplated that the stiffness of the elongate shaft 14 may be modified to form modulation systems 10 for use in various vessel diameters. The elongate shaft 14 may further include one or more lumens extending therethrough. For example, the elongate shaft 14 may include a guidewire lumen and/or one or more auxiliary lumens. The lumens may be configured in any suitable way such as those ways commonly used for medical device. For example, the guidewire lumen may extend the entire length of the elongate shaft 14 such as in an over-the-wire catheter or may extend only along a distal portion of the elongate shaft 14 such as in a single operator exchange (SOE) catheter. These examples are not intended to be limiting, but rather examples of some possible configurations. While not explicitly shown, the modulation system 10 may further include temperature sensors/wire, an infusion lumen, radiopaque marker bands, fixed guidewire tip, a guidewire lumen, external sheath and/or other components to facilitate the use and advancement of the system 10 within the vasculature may be incorporated.

The system 10 may further include one or more ultrasound transducers 22 disposed adjacent to the distal end 20 of the elongate shaft 14. The transducer 22 may have a proximal end 28 adjoining, or positioned adjacent to, the distal end 20 of the elongate shaft. The transducer 22 may extend distally from a proximal end 28 thereof for a length L and terminate at a distal end 30. The transducer 22 may have a first side surface 24 defined by the length L of the transducer and a height H of the transducer 22. The transducer 22 may also include a second side surface 26 also defined by the height H and length L of the transducer 22. The second side surface 26 may be generally opposite and facing approximately 180° from the first side surface 24. The first and second side surfaces 24,26 may be configured to radiate acoustic energy therefrom. The remaining surfaces (e.g. excluding surfaces 24,26) of the transducer 22 may form a perimeter of the transducer 22.

In some embodiments, the transducer 22 may be formed of a separate structure and attached to the elongate shaft 14. For example, the transducer 22 may be bonded or otherwise attached to the elongate shaft 14. In some instances, the transducer 22 may include a ring or other retaining or holding mechanism (not explicitly shown) disposed around the perimeter of the transducer 22. The transducer 22 may further include a post, or other like mechanism, affixed to the ring such that the post may be attached to the elongate shaft 14 or other member. In some instances, the ring may be attached to the transducer 22 with a flexible adhesive, such as, but not limited to, silicone. However, it is contemplated that the ring may be attached to the transducer 22 in any manner desired.

In some instances, the transducer 22 may be fixedly attached to the elongate shaft 14. In such cases, when it is desirable to rotate the transducer 22 it may be necessary to rotate the entire elongate shaft 14. As will be discussed in more detail below, it may not be necessary to rotate the elongate shaft 14 360° as the transducer 22 may emit acoustic energy in two directions simultaneously. For example, the transducer 22 may ablate an entire perimeter of a vessel by only rotating the transducer 22 and/or elongate shaft 14 180°. In other instances, the transducer 22 may be rotatably attached to the elongate shaft 14 such that the transducer 22 can rotate independently of the elongate shaft 14. For example, the transducer 22 may be coupled to a micromotor such that the transducer 22 may be rotated.

The transducer 22 may be formed from any suitable material such as, but not limited to, lead zirconate titanate (PZT). It is contemplated that other ceramic or piezoelectric materials may also be used. In some instances, the transducer 22 may include a layer of gold, or other conductive layer, disposed on the first and second surfaces 24, 26 over the PZT crystal for connecting electrical leads to the transducer 22. In some instances, one or more tie layers may be used to bond the gold to the PZT. For example, a layer of chrome may be disposed between the PZT and the gold to improve adhesion. In other instances, the transducer 22 may include a layer of chrome over the PZT followed by a layer of nickel, and finally a layer of gold. These are just examples. It is contemplated that the layers may be deposited on the PZT using sputter coating, although other deposition techniques may be used as desired.

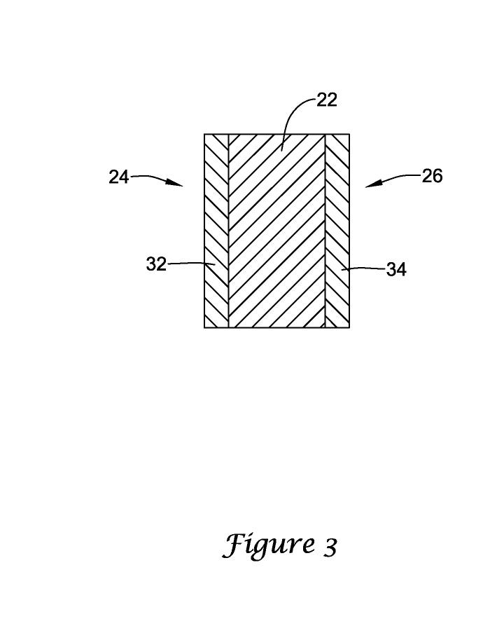

As shown in more detail in FIG. 3, the transducer 22 may further include a first matching layer 32 disposed on the first surface 24 and a second matching layer 34 disposed on the second surface 26. In some instances, the matching layers 32,34 may provide acoustic impedance matching for efficient transmission. In some instances, the matching layer material may be selected such that acoustic impedance of matching layer 32,34 is equal to the geometric mean of the acoustic impedance of the transducer 22 (e.g. PZT) and adjacent media (e.g. blood). In some instances, the matching layers 32,34 may be a silver filled epoxy, although other materials may be used as desired. The matching layers 32,34 may each have a thickness approximately equal to one-fourth of the operating frequency (e.g. wavelength), although other thicknesses may be used as desired.

It is contemplated that the faces 24,26 of the transducer 22 may take any shape desired, such as, but not limited to, square, rectangular, polygonal, circular, oblong, etc. The acoustic energy radiated from the transducer 22 may take the shape of the transducer 22 (e.g. a rectangular transducer 22 will generate a rectangular adhesion of approximately equal size to the transducer 22). Thus, the shape of the transducer 22 may be selected based on the desired treatment and the shape best suited for that treatment. It is contemplated that the transducer 22 may also be sized according to the desired treatment region. For example, in renal applications, the transducer 22 may be sized to be compatible with a 6 French guide catheter, although this is not required. The length L of the transducer 22 may be sized to allow the transducer 22 to navigate the passageways to the desired treatment region. In some instances, the transducer 22 may have a length L in the range of 0.5 to 10 millimeters (mm), 2-8 mm, or 3-6 mm. It is contemplated that, in certain applications, the transducer 22 may have a length less than 0.5 mm or greater than 10 mm. The height H of the transducer 22 may be dependent on the size of the guide catheter. For example, a transducer 22 for use with a 6 French guide catheter may have a height H of 1.5 mm or less. In some instances, the transducer 22 may be used without a guide catheter. As such, the height H of the transducer 22 may be limited by the desired treatment region. The width W of the transducer 22 may be determined by the sum of the thicknesses of the PZT crystal, tie layer(s), conductive layer(s), and the matching layers. In some instances, the thickness of the PZT crystal may be approximately equal to one-half the operating frequency (e.g. wavelength). In some embodiments, a transducer 22 including a PZT crystal and two matching layers 32,34 may have a thickness approximately equal to the operational frequency. However, the thickness of the transducer 22 may be less than or greater than the operational frequency as desired.

While not explicitly shown, the transducer 22 may be connected to a control unit (such as control unit 18 in FIG. 1) by electrical conductor(s). In some embodiments, the electrical conductor(s) may be disposed within a lumen of the elongate shaft 14. In other embodiments, the electrical conductor(s) may extend along an outside surface of the elongate shaft 14. The electrical conductor(s) may provide electricity to the transducer 22 which may then be converted into acoustic energy. The acoustic energy may be directed from the transducer 22 in a direction generally perpendicular to the surfaces 24,26 of the transducer 22, as illustrated by arrows 40 in FIG. 2. As discussed above, the acoustic energy radiated from the transducer 22 may take the shape of the transducer 22, e.g. a rectangular transducer will generate a rectangular adhesion having a size approximately equal to the size of the transducer 22. Thus, the acoustic energy may be radiated from the entire surface 24,26 and not an isolated point.

As discussed above, the transducer 22 may be formed with a matching layer 32,34 on two sides 24, 26 of the transducer 22. In the absence of an air backing layer, acoustic energy may be directed from both the first side surface 24 and the second side surface 26 simultaneously. This may allow two sides of a vessel to be ablated simultaneously. As such, the transducer 22 may perform the desired ablation twice as fast as an ultrasound transducer which includes a backing layer. In some instances, such as when circumferential ablation is desired, the transducer 22 and/or elongate shaft 14 may need to be rotated to complete the ablation. As two locations are being ablated simultaneously, the transducer 22 may only need to be rotated 180° to complete circumferential (360°) ablation. If multiple radial ablation points are desired, the transducer 22 only needs to rotated half as many times as in single direction ablation. In some instances, the transducer 22 and/or elongate shaft 14 may be manually rotated (e.g. by a physician). Limiting the degree of rotation of the modulation system 10 may allow the transducer 22 to be fixedly secured to the elongate shaft 14 or further facilitate manual rotation. However, in other instances, the transducer 22 may be rotated continuously and/or automatically using a micromotor or other rotating mechanism. In some instances, when the transducer 22 is spun continuously, the speed of rotation may be reduced due to simultaneous ablation. In some embodiments, the elongate shaft 14 may be longitudinally displaced to allow for ablation along a length of a vessel. For example, the modulation system 10 may be advanced within a vessel to a desired location and energy supplied to the transducer 22. Once ablation at the location has been completed, the transducer 22 may be longitudinally displaced and energy again supplied to the transducer 22. The transducer 22 may be longitudinally and/or radially displaced as many times as necessary to complete the desired treatment. It is further contemplated that multiple transducers 22 may be placed along the longitudinal axis or radially offset to minimize the number of times the modulation system 10 needs to be displaced. For example, the transducers 22 may be placed in phased arrays and/or geometric focusing arrays depending on the desired application.

In some instances, it may be desirable to center the transducer 22 within the vessel being treated. Locating the transducer 22 in the center of the vessel may allow blood flow to pass by both surfaces 24,26. This may provide passive cooling to the transducer 22 during operation. It is contemplated that a two-sided transducer 22 may be cooled more efficiently than a one-sided transducer. The backing layer, which is absent in the present transducer 22, may prevent the back side of the one-sided transducer from benefiting from the passive cooling supplied by the blood flow. Increased cooling (by allowing both surfaces 24,26 to contact fluid flow) may increase the efficiency of the transducer 22. As the power is relayed to the transducer 22, the power that does not go into generating acoustic power generates heat. As the transducer 22 heats, it becomes less efficient, thus generating more heat. Passive cooling provided by the flow of blood may help improve the efficiency of the transducer 22. As such, additional cooling mechanisms may not be necessary. However, in some instances, additional cooling may be provided by introducing a cooling fluid to the modulation system.

In order to allow blood to pass by both sides of the transducer 22 a centering mechanism may be provided. In some instances, an inflatable balloon may be provided. The inflatable balloon may be provided along the elongate shaft 14. When the desired treatment area is reached, the inflatable balloon may be expanded. It is contemplated that the inflatable balloon be sized and shaped to allow blood flow to continue to pass the transducer 22. For example, the balloon may only partially occlude the vessel. Alternatively, in some embodiments, a spacing basket or struts may be used to center the system 10 within the vessel.

It is further contemplated that in some instances two sided ultrasound ablation may utilize energy more efficiently than one-sided ablation. For example, allowing acoustic energy to radiate from two sides may reduce energy lost when the ultrasound waves are reflected off of a backing layer of a one-sided transducer. Increased cooling (by cooling at both sides) of the two-sided transducer 22 may also contribute to increased efficiency.

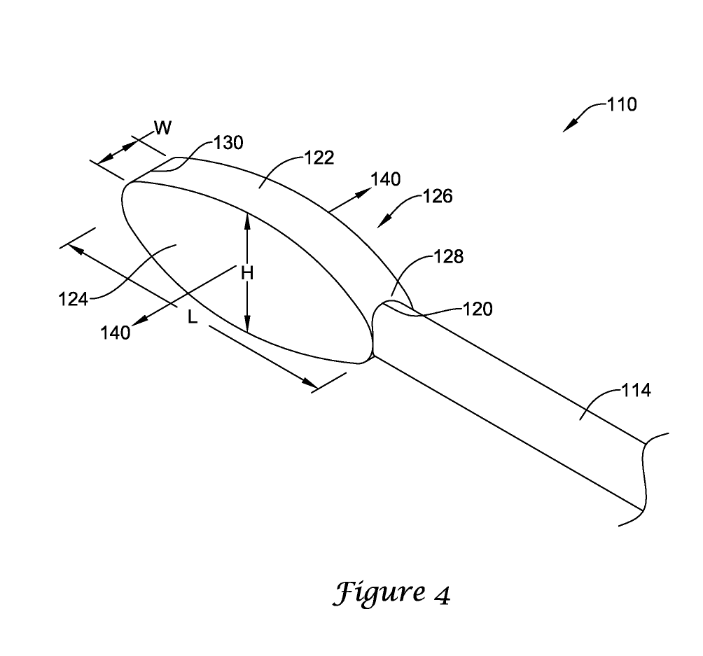

FIG. 4 is a perspective view of a distal end of another illustrative renal nerve modulation system 110 that may be similar in form and function to other systems disclosed herein. The system 110 may include an elongate shaft 114 having a distal end 120. The elongate shaft 114 may extend proximally from the distal end 120 to a proximal end configured to remain outside of a patient's body.

The system 110 may further include one or more ultrasound transducers 122 disposed adjacent to the distal end 120 of the elongate shaft 114. The transducer 122 may be positioned parallel to a longitudinal axis of the elongate shaft 114. The transducer 122 may have a proximal end 128 adjoining, or positioned adjacent to, the distal end 120 of the elongate shaft. The transducer 122 may extend distally from a proximal end 128 thereof for a length L and terminate at a distal end 130. The transducer 122 may have a first side surface 124 extending along the length L of the transducer 122. The first side surface may have a generally oval shape and have a maximum height H. The transducer 122 may also include a second side surface 126 having a similar shape to the first side surface 126 and defined by the height H and length L of the transducer 122. The second side surface 126 may be generally opposite and facing approximately 180° from the first side surface 124. The first and second side surfaces 124,126 may be configured to radiate acoustic energy therefrom. The remaining surfaces (e.g. excluding surfaces 124,126) of the transducer 122 may form a perimeter of the transducer 122. The acoustic energy may be directed from the transducer 122 in a direction generally perpendicular to the surfaces 124,126 of the transducer 122, as illustrated by arrows 140 in FIG. 4.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A system for nerve modulation, comprising
an elongate shaft having a proximal end region and a distal end region; and
an ultrasound transducer positioned adjacent the distal end region;
wherein the ultrasound transducer is configured to radiate acoustic energy in two directions simultaneously.

2. The system of claim 1, wherein the transducer comprises a lead zirconate titanate (PZT) crystal.

3. The system of claim 1, wherein the transducer includes a first side surface and a second side surface.

4. The system of claim 3, wherein the acoustic energy is radiated from the first and second side surfaces.

5. The system of claim 4, wherein the first side surface faces a first direction and wherein the second side surface faces a second direction opposite the first direction.

6. The system of claim 1, wherein the transducer includes a perimeter.

7. The system of claim 6, further comprising a retaining mechanism disposed about the perimeter.

8. The system of claim 7, wherein the retaining mechanism is fixedly secured to the distal end region of the elongate shaft.

9. The system of claim 7, wherein the retaining mechanism is rotatably secured to the distal end region of the elongate shaft.

10. The system of claim 3, further comprising a matching layer disposed on the first side surface and the second side surface.

11. The system of claim 10, wherein the matching layer comprises a silver filled epoxy.

12. An intravascular nerve ablation system comprising
an elongate shaft having a proximal end and distal end and a lumen extending therebetween;
an ultrasound transducer positioned adjacent to the distal end of the elongate shaft, the transducer including a first side surface and a second side surface, the second side surface facing 180° from the first side surface;
a retaining ring disposed about a perimeter of the transducer; and
a post secured to the retaining ring.

13. The system of claim 12, wherein the transducer comprises a lead zirconate titanate (PZT) crystal and a gold coating on the first and second side surfaces.

14. The system of claim 13, further comprising a tie layer disposed between the PZT crystal and the gold coating.

15. The system of claim 12, wherein the transducer is configured to radiate acoustic energy from the first side surface and the second side surface simultaneously.

16. The system of claim 15, wherein the acoustic energy is radiated having a shape similar to the shape of the transducer.

17. The system of claim 12, wherein the first and second side surfaces have a rectangular shape.

18. The system of claim 12, wherein the first and second side surfaces have an oval shape.

19. The system of claim 12, further comprising a matching layer disposed on the first and second side surfaces.

20. An intravascular nerve ablation system comprising
an elongate shaft having a proximal end and distal end;
an ultrasound transducer having a proximal end, the proximal end of the transducer positioned adjacent to the distal end of the elongate shaft, the transducer including a first side surface and a second side surface, the second side surface facing 180° from the first side surface;
a first matching layer disposed on the first side surface;
a second matching layer disposed on the second side surface;
a retaining ring disposed about a perimeter of the transducer; and
a post extending between the distal end of the elongate shaft and the proximal end of the transducer;
wherein the transducer is configured to radiate acoustic energy from the first side surface and the second side surface.
